(19) United States
(12) Patent Application Publication
(10) Pub. No.: US 2018/0153382 A1
(43) Pub. Date: Jun. 7, 2018

(54) OPTICAL SCANNING ENDOSCOPE AND OPTICAL FIBER SCANNING APPARATUS

(71) Applicant: OLYMPUS CORPORATION, Tokyo (JP)

(72) Inventor: Masanori OGATA, Matsumoto-shi (JP)

(73) Assignee: OLYMPUS CORPORATION, Tokyo (JP)

(21) Appl. No.: 15/877,428

(22) Filed: Jan. 23, 2018

Related U.S. Application Data
(63) Continuation of application No. PCT/JP2015/070973, filed on Jul. 23, 2015.

(51) Int. Cl. A61B 1/00; A61B 1/07

(52) U.S. Cl. CPC A61B 1/00172; A61B 1/07

(57) ABSTRACT

An optical fiber scanning apparatus includes a tubular housing, an optical fiber disposed along a Z axis, which is a center axis of the housing, provided with a magnet, and configured to emit light from a free end thereof, an X-axis coil including at least one spiral coil configured to apply a magnetic field in an X-axis direction orthogonal to the Z axis, a Y-axis coil including at least one spiral coil configured to apply a magnetic field in a Y-axis direction orthogonal to the Z axis and the X axis, and a magnetic-field inducing section including an inner magnetic-field inducing section including a core disposed in a center portion on an inside, which is the center axis side of the spiral coils, and an outer magnetic-field inducing section disposed on an outside opposite to the inside.

### CROSS REFERENCE TO RELATED APPLICATION

This application is a continuation application of PCT/JP2015/070973 filed on Jul. 23, 2015, the entire contents of which are incorporated herein by this reference.

### BACKGROUND OF INVENTION

### 1. Field of the Invention

The present invention relates to an optical fiber scanning apparatus including a housing provided with a magnetic-field generating unit and an optical fiber configured to emit light from a free end and provided with a permanent magnet and an optical scanning endoscope including the optical fiber scanning apparatus at a distal end portion of an insertion section.

### 2. Description of the Related Art

An image pickup apparatus including an image pickup device such as a CCD or a CMOS image sensor simultaneously receives, with a large number of light receiving elements arranged in matrix, reflected light reflected from a subject and acquires an object image. In the case of an endoscope that photographs a dark inside of a body, the endoscope acquires an image in a range illuminated by light from a light source.

On the other hand, an optical scanning image pickup apparatus sequentially receives, while scanning and irradiating an object with a light spot, reflected light of the light spot and creates an object image on the basis of data of the received light.

For example, in the optical scanning image pickup apparatus, an optical fiber scanning apparatus two-dimensionally scans a free end of an optical fiber in cantilevered state, which guides light from a light source, to perform the scan irradiation of the light spot.

The optical fiber scanning apparatus performs the optical fiber scanning by, for example, applying a magnetic force of a magnetic-field generating unit to an optical fiber provided with a magnet.

Japanese Patent Application Laid-Open No. 2014-81484 discloses an optical fiber scanning apparatus that uses a magnetic force. In this conventional optical fiber scanning apparatus, in a cylinder, an optical fiber provided with a permanent magnet is disposed along a center axis of a magnetic-field generating unit including four plane spiral coils, which are orthogonally disposed or disposed to be opposed to one another.

### SUMMARY OF THE INVENTION

An optical fiber scanning apparatus in an embodiment includes: a tubular housing; an optical fiber disposed along a Z axis, which is a center axis of the housing, provided with a permanent magnet, and configured to emit light from a free end thereof; an X-axis coil including at least one spiral coil configured to apply a magnetic field in an X-axis direction orthogonal to the Z axis to the permanent magnet and scan the free end in the X-axis direction; a Y-axis coil including at least one spiral coil configured to apply a magnetic field in a Y-axis direction orthogonal to the Z axis and the X axis to the permanent magnet and scan the free end in the Y-axis direction; and a magnetic-field inducing section including an inner magnetic-field inducing section including a core disposed in a center portion on an inside, which is the center axis side of each of the spiral coils included in the x-axis coil and the y-axis coil, and configured to induce the magnetic field in a direction perpendicular to the center axis of the housing and an outer magnetic-field inducing section disposed on an outside opposite to the inside of each of the spiral coils.

An optical scanning endoscope in another embodiment includes, at a distal end portion of an insertion section, an optical fiber scanning apparatus which includes: a tubular housing; an optical fiber disposed along a Z axis, which is a center axis of the housing, provided with a permanent magnet, and configured to emit light from a free end thereof; an X-axis coil including at least one spiral coil configured to apply a magnetic field in an X-axis direction orthogonal to the Z axis to the permanent magnet and scan the free end in the X-axis direction; a Y-axis coil including at least one spiral coil configured to apply a magnetic field in a Y-axis direction orthogonal to the Z axis and the X axis to the permanent magnet and scan the free end in the Y-axis direction; and a magnetic-field inducing section including an inner magnetic-field inducing section including a core disposed in a center portion on an inside, which is the center axis side of each of the spiral coils included in the x-axis coil and the y-axis coil, and configured to induce the magnetic field in a direction perpendicular to the center axis of the housing and an outer magnetic-field inducing section disposed on an outside opposite to the inside of each of the spiral coils.

### BRIEF DESCRIPTION OF THE DRAWINGS

FIG. 1 is a sectional view taken along a center axis of an optical fiber scanning apparatus in a first embodiment;

FIG. 2 is a sectional view of the optical fiber scanning apparatus in the first embodiment taken along a II-II line in FIG. 1;

FIG. 3 is an exploded view of a main part of the optical fiber scanning apparatus in the first embodiment;

FIG. 4A is a top view of a coil chip of the optical fiber scanning apparatus in the first embodiment;

FIG. 4B is a sectional view of the coil chip of the optical fiber scanning apparatus in the first embodiment;

FIG. 5A is a schematic diagram for explaining operation of the optical fiber scanning apparatus in the first embodiment;

FIG. 5B is a schematic diagram for explaining the operation of the optical fiber scanning apparatus in the first embodiment;

FIG. 6A is a schematic diagram showing a magnetic field of a conventional optical fiber scanning apparatus;

FIG. 6B is a schematic diagram showing a magnetic field of the optical fiber scanning apparatus in the first embodiment;

FIG. 7 is a sectional view of an optical fiber scanning apparatus in a modification 1 of the first embodiment;

FIG. 8 is a sectional view of an optical fiber scanning apparatus in a modification 2 of the first embodiment;

FIG. 9 is a sectional view of an optical fiber scanning apparatus in a modification 3 of the first embodiment;

FIG. 10 is a sectional view of an optical fiber scanning apparatus in a second embodiment;

FIG. 11A is a top view of a coil chip of an optical fiber scanning apparatus in the second embodiment;

FIG. 11B is a sectional view of the coil chip of the optical fiber scanning apparatus in the second embodiment;

FIG. 12 is a sectional view of a coil chip of an optical fiber scanning apparatus in a modification 1 of the second embodiment;

FIG. 13 is a top view of a coil chip of an optical fiber scanning apparatus in a modification 2 of the second embodiment;

FIG. 14 is a top view of a coil chip of an optical fiber scanning apparatus in a modification 3 of the second embodiment;

FIG. 15 is a top view of a coil chip of an optical fiber scanning apparatus in a modification 4 of the second embodiment;

FIG. 16 is a sectional view of the coil chip of the optical fiber scanning apparatus in the modification 4 of the second embodiment;

FIG. 17 is a sectional view of a coil chip of an optical fiber scanning apparatus in a third embodiment;

FIG. 18 is a sectional view of a coil chip of an optical fiber scanning apparatus in a fourth embodiment; and

FIG. 19 is an exterior view of an optical scanning endoscope in a fifth embodiment.

### DETAILED DESCRIPTION OF THE PREFERRED EMBODIMENTS

### First Embodiment

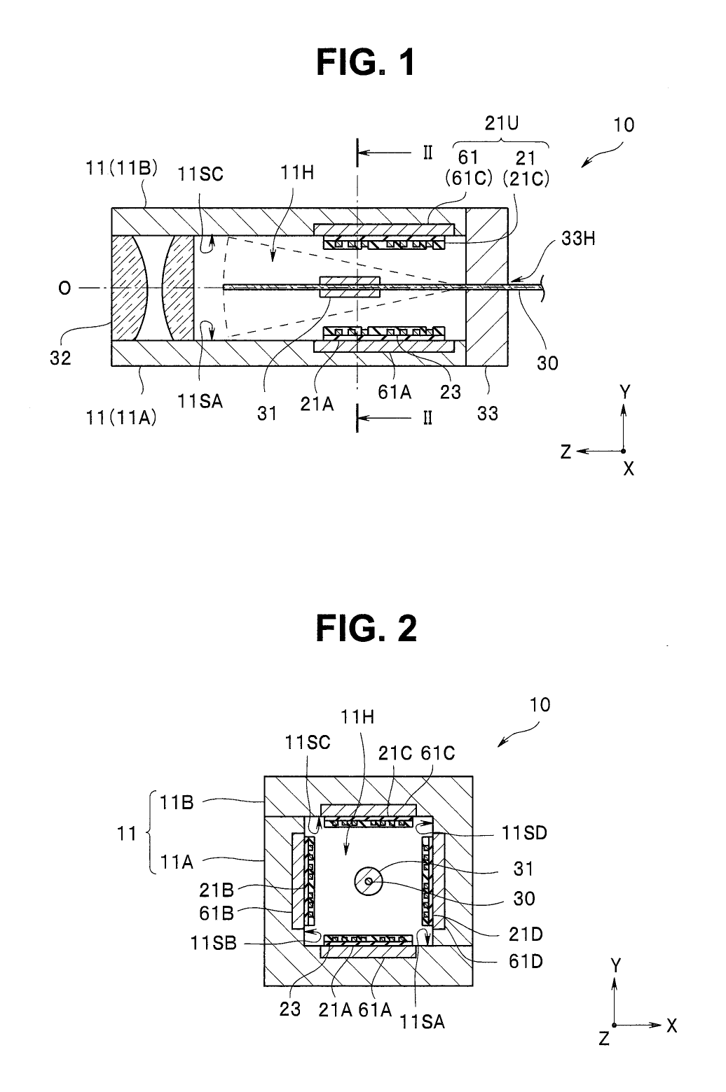

An optical fiber scanning apparatus 10 in a first embodiment is explained with reference to FIG. 1 to FIG. 4B. Note that, in the following explanation, drawings based on respective embodiments are schematic and relations between thicknesses and widths of respective portions, ratios of the thicknesses of the respective portions, and the like are different from actual ones. Portions having different relations and ratios of dimensions one another are sometimes included among the drawings. Illustration of a part of components and imparting of reference numerals and signs are sometimes omitted.

Am optical fiber scanning apparatus 10 includes a housing 11, an optical fiber 30, a magnetic-field generating unit 21U, and an illumination optical system 32. A hollow section 11H is present in the housing 11 having a tubular shape. The optical fiber 30 is disposed in a cantilevered state along a center axis O in a major axis direction (a Z direction) of the hollow section 11H of the housing 11. The magnetic-field generating unit 21U includes coil chips 21A to 21D and outer yokes 61A to 61D disposed on an outside of the coil chips 21A to 21D. Note that the outside is an opposite side of an inside, which is the center axis O side of the housing 11.

The optical fiber 30 guides light from a light source unit (not shown in the figure) and emits illumination light from a free end. The illumination light spot-irradiates an object via the illumination optical system 32 including a plurality of lenses.

A permanent magnet 31 is fixed to the optical fiber 30. The permanent magnet 31 made of, for example, a SmCo alloy is tubular and is magnetized in a longitudinal direction. The optical fiber 30 is inserted through a through-hole 33H of a holding member 33 and is bonded to the holding member 33. The free end of the optical fiber 30 in the cantilevered state, a bonding section of which to the holding member 33 is fixed, is movable in an XY plane upward and downward and to the left and the right with a fixed end as a base point.

The housing 11, that is, a first housing 11A and a second housing 11B are made of nonmagnetic metal such as aluminum or resin.

In the housing 11, the hollow section 11H, a cross section (the XY plane) of which orthogonal to the center axis O is square, is present. The housing 11 includes the first housing 11A and a second housing 11B bonded to the first housing 11A. As the bonding, an adhesive may be used. However, since a gap is formed, it is desirable to directly fasten the first housing 11A and the second housing 11B using screws or the like not via other members.

The housing 11 has a function of a positioning member for accurately disposing the magnetic-field generating unit 21U. Thickness of a wall of the housing 11 may be small if the wall has a certain degree of strength. The thickness of the wall of the housing 11 is shown large in FIG. 2 and the like. However, the thickness is, for example, 10 μm or more and 1000 μm or less. If the thickness is within the range, it is easy to reduce the housing 11 in size (in diameter) while guaranteeing the function of the positioning member.

A first surface 11SA and a second surface 11SB orthogonal to the first surface 11SA are present on an inside of the first housing 11A. A third surface 11SC and a fourth surface 11SD orthogonal to the third surface 11SC are present on an inside of the second housing 11B. When the first housing 11A and the second housing 11B are bonded, the first surface 11SA to the fourth surface 11SD configure an inner surface of the hollow section 11H of the housing 11.

All of corner portions of a cross section of the hollow section 11H formed by bonding the second surface 11SB and the third surface 11SC to be orthogonal and bonding the first surface 11SA and the fourth surface 11SD to be orthogonal are 90 degrees. A shape of the first housing 11A and a shape of the second housing 11B are set such that lengths of four sides of the cross section of the hollow section 11H formed when the first housing 11A and the second housing 11B are bonded are equal.

The magnetic-field generating unit 21U including the outer yokes 61A to 61D is disposed in the hollow section 11H of the housing 11. That is, the outer yoke 61A and the coil chip 21A are disposed on the first surface 11SA of the first housing 11A. The outer yoke 61B and the coil chip 21B are disposed on the second surface 11SB of the first housing 11A. The outer yoke 61C and the coil chip 21C are disposed on the third surface 11SC of the second housing 11B. The outer yoke 61D and the coil chip 21D are disposed on the fourth surface 11SD of the second housing 11B. Note that, in the following explanation, when each of a plurality of components is referred to, one alphabet character at an end of a sign is sometimes omitted. For example, each of the coil chips 21A to 21D is referred to as coil chip 21.

The outer yoke 61 disposed on an outside of the coil chip 21 is a magnetic-field inducing section made of a permalloy (a NiFe alloy) that induces magnetic fields generated by the coil chips 21A to 21D. The outer yoke 61 is desirably made of a soft magnetic material, specific permeability of which at a driving frequency of the magnetic-field generating unit 21U is 100 or more, such as iron, cobalt, nickel, a permalloy, soft ferrite, or an amorphous alloy.

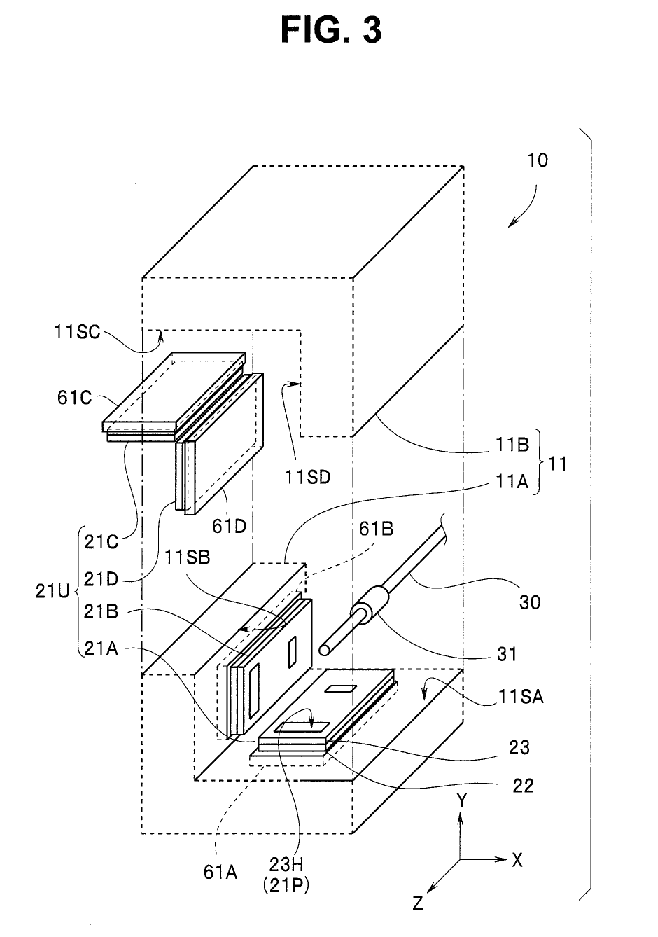

The outer yoke 61 completely covers one surface of a plane coil 21S. An effect of inducing a magnetic field is conspicuous if thickness of the outer yoke 61 is 1 μm or more. An upper limit of the thickness of the outer yoke 61 is determined according to, for example, intensity of a magnetic field generated by the coil chip 21. The upper limit is, for example, 10 μm to 1000 μm. In FIG. 3 and the like, the outer yoke 61 completely covers the plane coil 21S. However, the outer yoke 61 may partially cover the plane coil 21S.

Note that, in the optical fiber scanning apparatus 10, the outer yoke 61 of a substantially rectangular parallelepiped is fit in a concave section of the housing 11. However, the outer yoke 61 may be bonded to an inner surface of the housing 11.

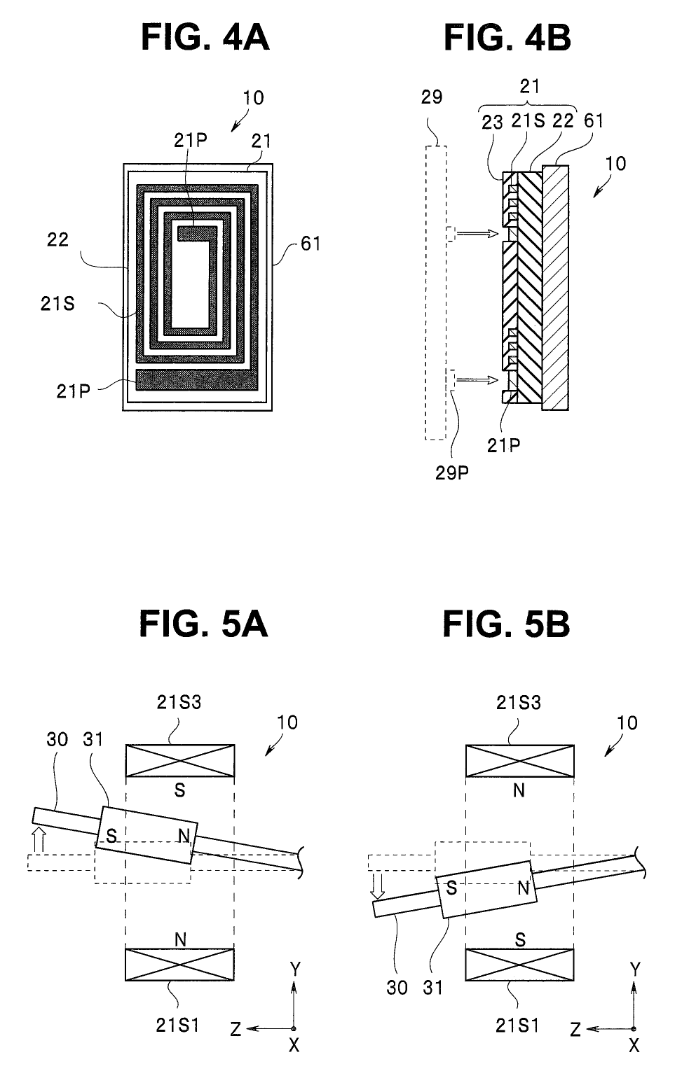

As shown in FIG. 4A and the like, in the coil chip 21, the plane coil 21S, which is a driving coil having a spiral shape, is disposed on a substrate 22 made of silicon via an insulating layer (not shown in the figure) of oxide silicon or the like.

The plane coil 21S is covered by an insulating layer 23 made of resin such as polyimide or epoxy except contact holes section in upper parts of bonding pads 21P at both ends. The illustrated plane coil 21S includes a conductor layer (a plane coil) made of patterned low-resistance metal such as copper or gold and an insulating layer that covers the conductor layer. Note that a bonding pad is also disposed in a coil center in the coil chip 21. However, in order to provide a bonding pad in a periphery of the coil, the coil chip 21 may further include an insulating layer of one layer/a draw-out wire or may be a multilayer coil obtained by stacking a plurality of plane coils via insulating layers as explained below.

The coil chip 21 can be manufactured by disposing a large number of plane coils on a silicon wafer and thereafter singulating the plane coils according to a MEMS semiconductor process. For example, it is possible to easily manufacture a large number of the coil chips 21 including high precision plane coils 21S by patterning with an additive method, a subtract method, or the like using a high precision resist mask manufactured by a photolithography method in which a photoresist and a photomask are used.

Note that, in the optical fiber scanning apparatus 10, the first housing 11A and the second housing 11B have substantially the same shape. The coil chips 21A to 21D have the same configuration. Therefore, in the optical fiber scanning apparatus 10, the first housing 11A and the second housing 11B can be manufactured in the same process and the coil chips 21A to 21D can be manufactured in the same processing. Further, the first housing 11A provided with the coil chips 21A and 21B and the second housing 11B provided with the coil chips 21C and 21D can be manufactured in the same process. Therefore, the optical fiber scanning apparatus 10 is easily manufactured.

As shown in FIG. 4B, two bonding pads 21P are bonded to, for example, by solder, an electrode 29P of a flexible wiring board 29 disposed on an inner surface side of the coil chip 21. Note that, in the drawings other than FIG. 4B, illustration of the flexible wiring board 29 is omitted. The flexible wiring board 29 is connected to a power supply unit (not shown in the figure), which supplies a driving current, by a not-shown wire.

The plane coil 21S generates a magnetic field in a direction orthogonal to a principal plane of the coil chip 21 when the driving current is applied to the bonding pad 21P. Intensity of the magnetic field is set by a current value of the driving current, the number of windings (the number of turns) of a spiral coil, and the like. When a direction of the driving current flowing in the coil is reversed, a direction of the generated magnetic field is reversed.

In the optical fiber scanning apparatus 10, plane coils 21S1 and 21S2 are respectively disposed on the first surface 11SA and the second surface 11SB orthogonal to each other of the first housing 11A. Plane coils 21S3 and 21S4 are respectively disposed on the third surface 11SC and the fourth surface 11SD orthogonal to each other of the second housing 11B. That is, the plane coil 21S1 and the plane coils 21S3 are disposed in opposed positions. The plane coil 21S2 and the plane coil 21S4 are disposed in opposed positions.

Therefore, the plane coils 21S1 and 21S3 generate magnetic fields in a Y-axis direction. The plane coils 21S2 and 21S4 generate magnetic fields in an X-axis direction. The optical fiber 30 (the permanent magnet 31) is disposed at an equal distance from the four plane coils 21S1 to 21S4, that is, in a center of the hollow section 11H of the housing 11.

A driving method for the optical fiber scanning apparatus 10 is briefly explained.

As shown in FIG. 5A, when a driving current is supplied to the plane coil 21S1, for example, a magnetic field having an N pole on an inner surface side is generated. At the same time, when a driving current is supplied to the plane coil 21S3, for example, a magnetic field having an S pole on an inner surface side is generated. That is, the plane coil 21S1 and the plane coil 21S3 disposed to be opposed to each other generate magnetic fields having different magnetic poles on the inner surface sides.

Therefore, a rear end side (an N pole) of the permanent magnet 31 disposed in the magnetic fields is lifted in a Y-axis upward direction. Therefore, the free end of the optical fiber 30 also moves in the Y-axis upward direction.

On the other hand, as shown in FIG. 5B, when a driving current in an opposite direction of a direction in the case of FIG. 5A is supplied to the plane coil 21S1, a magnetic field having an S pole on an inner surface side is generated. Similarly, when a driving current in an opposite direction of a direction in the case of FIG. 5A is supplied to the plane coil 21S3, a magnetic field having an N pole on an inner surface side is generated. Then, the rear end (the N pole) of the permanent magnet 31 disposed in the magnetic fields is lowered in a Y-axis downward direction. Therefore, the free end of the optical fiber 30 also moves in the Y-axis downward direction.

The free end of the optical fiber 30 is scanned in the Y-axis direction by controlling the directions of the driving currents supplied to the plane coils 21S1 and 21S3. Similarly, the free end of the optical fiber 30 is scanned in the X-axis direction by controlling the directions of the driving currents supplied to the plane coils 21S2 and 21S4.

Note that the permanent magnet 31, the optical fiber, or the magnetic-field generating unit 21U may be disposed such that a magnetic field is applied to a distal end side of the permanent magnet 31. For example, two-dimensional scanning is possible even if only two plane coils 21S1 and 21S2 are driven. Further, in the case of an optical fiber scanning apparatus in which only one-dimensional scanning is necessary, scanning is possible with only one plane coil 21S.

By controlling the directions of the driving currents supplied to the four plane coils 21S1 to 21S4, the free end of the optical fiber 30 is two-dimensionally scanned in the XY plane. A scanning width is controlled by a driving current value. As a result, a light spot emitted from the free end of the optical fiber 30 is two-dimensionally scanned.

As a two-dimensional scanning system, a spiral scanning system, a raster scanning system, or a Lissajous system is desirable because image processing is easy. The raster scanning system is particularly desirable because illumination can be uniformly performed.

The optical fiber scanning apparatus 10 is small in diameter because the magnetic-field generating unit 21U is configured from extremely thin coil chips 21A to 21D having thickness of, for example, 10 μm or more and 500 μm or less. Further, the coil chips 21A to 21D are respectively disposed on the inner surface of the hollow section 11H, the cross section of which is square, of the housing 11. Therefore, the coil chips 21A to 21D are accurately disposed.

Therefore, the optical fiber scanning apparatus 10 is small in diameter and can perform high precision scan irradiation.

Further, the optical fiber scanning apparatus 10 includes a magnetic-field inducing section, which includes the outer yoke 61 made of the soft magnetic material, configured to induce a magnetic field generated by the plane coil 21S.

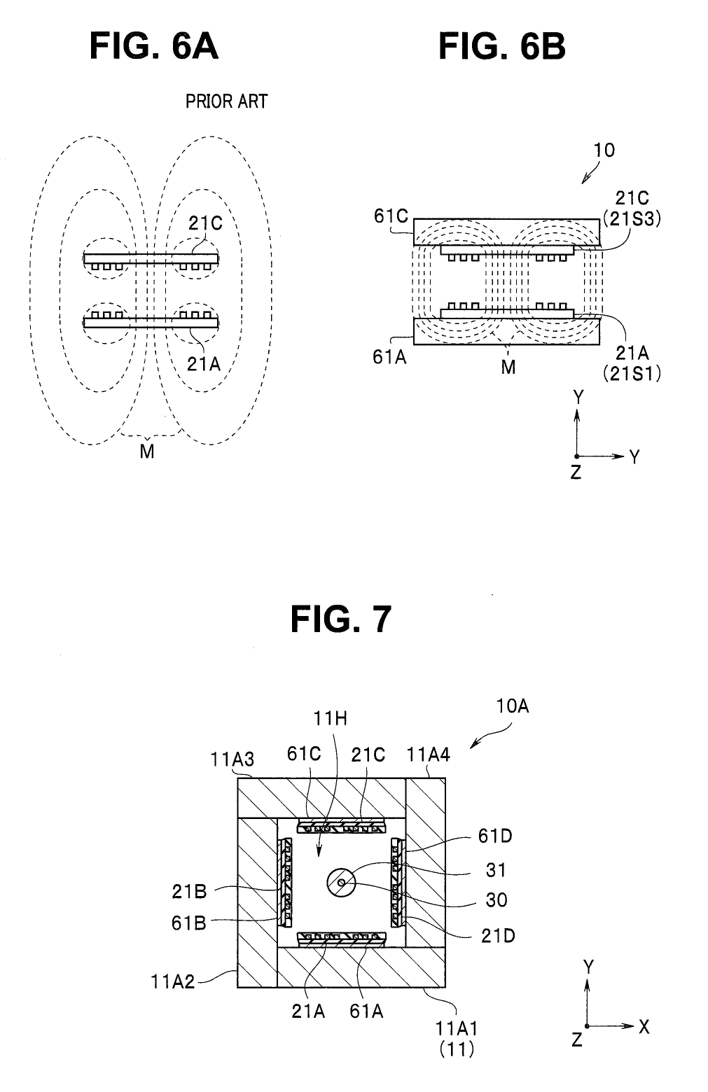

As shown in FIG. 6A, in a conventional optical fiber scanning apparatus, a magnetic field M generated by the plane coil 21S spreads large and a magnetic field leaks to an outside of a magnetic-field generating unit.

On the other hand, in the optical fiber scanning apparatus 10 shown in FIG. 6B, the magnetic field M generated by the plane coil 21S is induced to the outer yoke 61. Therefore, the magnetic field M generated by the plane coil 21S less easily leaks to an outside of the magnetic-field generating unit 21U. The magnetic field M can be concentrated on a vicinity of the permanent magnet 31. Therefore, the optical fiber scanning apparatus 10 can perform efficient scan irradiation. That is, the magnetic field leaking to the outside of the optical fiber scanning apparatus is taken into an inside by the outer yoke 61. Therefore, it is possible to concentrate the magnetic field on the vicinity of the permanent magnet 31. It is possible to efficiently use a magnetic field generated from the magnetic-field generating unit 21U.

### Modifications of the First Embodiment

Optical fiber scanning apparatuses 10A to 10C in modifications 1 to 3 of the first embodiment are explained. Since the optical fiber scanning apparatuses 10A to 10C are similar to the optical fiber scanning apparatus 10, components having the same functions are denoted by the same reference numerals and signs and explanation of the components is omitted. Note that, in the figures referred to below, an optical fiber, a magnetic-field generating unit, and the like are sometimes not illustrated.

The optical fiber scanning apparatuses 10A to 10C have the effects of the optical fiber scanning apparatus 10 and further have characteristic effects.

### Modification 1 of the First Embodiment

As shown in FIG. 7, in the optical fiber scanning apparatus 10A in the modification 1, the outer yokes 61A to 61D are made of soft magnetic foil bonded to the inner surface of the housing 11. The housing 11 is configured from four housings 11A1 to 11A4.

Like the outer yoke 61, the outer yokes 61A to 61D are made of a soft magnetic material such as iron, cobalt, nickel, a permalloy, or an amorphous alloy. However, thicknesses of the outer yokes 61A to 61D are relatively as small as, for example, 1 μm to 50 μm. Therefore, it is unnecessary to form the concave section in the housing 11.

By bonding the soft magnetic foil to a rear surface of a silicon wafer provided with a large number of coil chips 21 and then singulating the soft magnetic foil, it is possible to easily manufacture a large number of the coil chips 21 to which the outer yokes 61A are bonded. Alternatively, a soft magnetic film may be formed on the rear surface of the silicon wafer by a sputtering method, an evaporation method, a plating method, or the like.

The optical fiber scanning apparatus 10A is more easily manufactured than the optical fiber scanning apparatus 10.

### Modification 2 of the First Embodiment

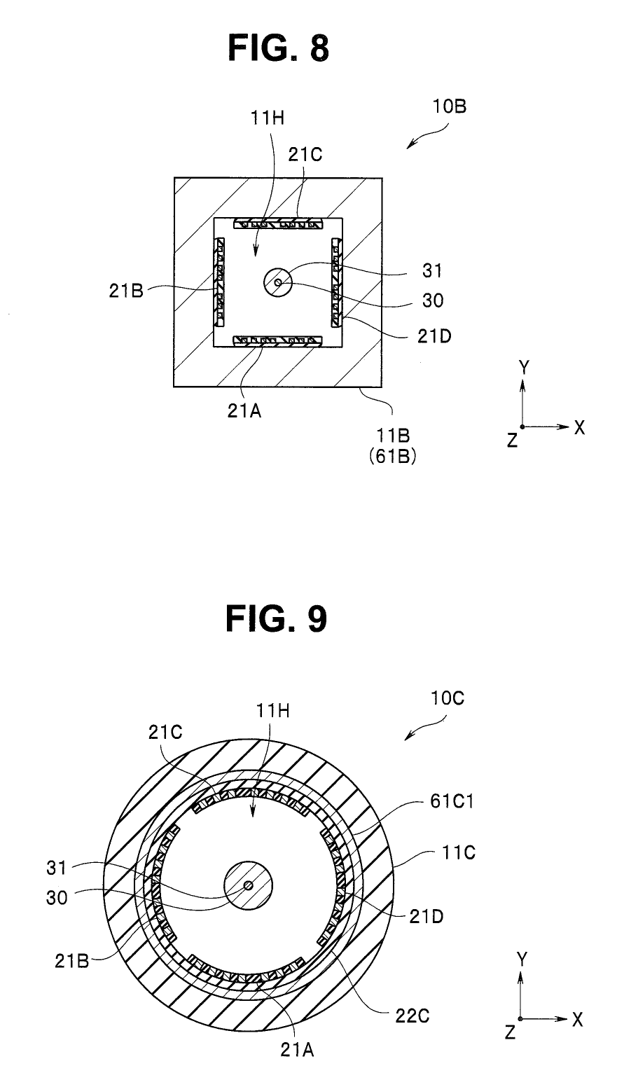

As shown in FIG. 8, in the optical fiber scanning apparatus 10B in the modification 2, a housing 11B made of a soft magnetic material has a function of the outer yoke 61B.

Like the outer yoke 61, the housing 11B is made of a soft magnetic material such as iron, cobalt, nickel, a permalloy, soft ferrite, or an amorphous alloy.

In the optical fiber scanning apparatus 10B, it is unnecessary to bond an outer yoke to the housing. Therefore, the optical fiber scanning apparatus 10B is more easily manufactured and smaller in diameter than the optical fiber scanning apparatus 10. Note that, in order to further reduce the diameter, the coil chip 21 may be fit in a concave section on an inner surface of the housing 11B.

### Modification 3 of the First Embodiment

As shown in FIG. 9, in the optical fiber scanning apparatus 10C in the modification 3, a housing 11C has a cylindrical shape. That is, the housing does not need to be a rectangular parallelepiped and may be a substantially rectangular parallelepiped, corner portions of an outer surface of which are curved or chamfered. A substrate 22C of the coil chips 21A to 21D disposed on an inner surface of the cylindrical housing 11C is made of, for example, polyimide having flexibility.

An outer yoke 61C of the optical fiber scanning apparatus 10C is a thin belt or a thin film made of a soft magnetic material disposed on the inner surface of the housing 11C. Note that, as in the optical fiber scanning apparatus 10B, the housing 11C may be configured by a soft magnetic material. A function of an outer yoke may be imparted to the housing 11C.

The optical fiber scanning apparatus 10C is more easily manufactured and smaller in diameter than the optical fiber scanning apparatus 10.

### Second Embodiment

An optical fiber scanning apparatus 10D in a second embodiment is explained. Since the optical fiber scanning apparatus 10D is similar to the optical fiber scanning apparatus 10 and the like, components having the same functions are denoted by the same reference numerals and signs and explanation of the components is omitted.

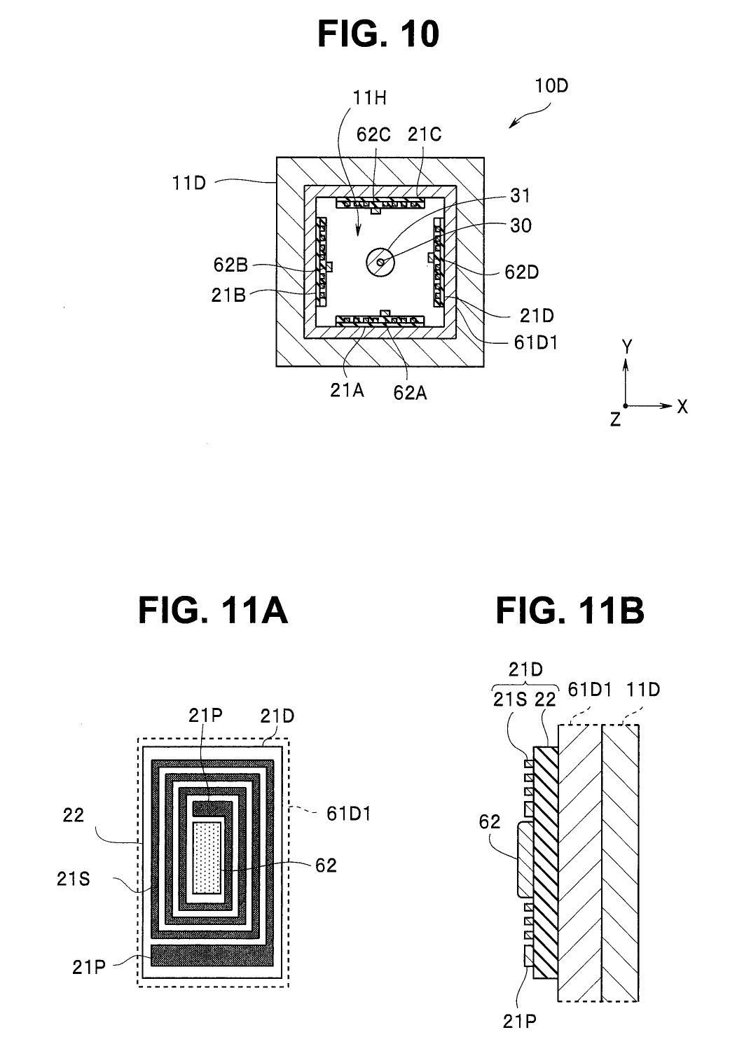

As shown in FIG. 10 to FIG. 11B, as a magnetic-field inducing section made of a soft magnetic material that induces a magnetic field generated by the coil chip 21, the optical fiber scanning apparatus 10D includes, in addition to an outer yoke 61D1, cores 62A to 62D, which are inner magnetic-field inducing sections, disposed on insides of the coil chips 21A to 21D.

The core 62 is disposed in a center portion of the plane coil 21S of the coil chip 21. Like the outer yoke 61, the core 62 is made of a soft magnetic material such as iron, cobalt, nickel, a permalloy, soft ferrite, or an amorphous alloy.

Thickness of the core 62 is, for example, 10 μm or more and 1000 μM or less. As the core 62, a thin belt having a predetermined shape may be bonded in the center portion of the coil chip 21. A soft magnetic film may be formed and patterned on the silicon wafer provided with the large number of the coil chips 21.

The optical fiber scanning apparatus 10D including the core 62 has the effects of the optical fiber scanning apparatus 10 and the like. The optical fiber scanning apparatus 10D can further concentrate the magnetic field M generated by the plane coil 21S on the vicinity of the permanent magnet 31. Therefore, the optical fiber scanning apparatus 10D can perform more efficient scan irradiation than the optical fiber scanning apparatus 10 and the like.

### Modifications of the Second Embodiment

Optical fiber scanning apparatuses 10E to 10H in modifications 1 to 4 of the second embodiment are explained. Since the optical fiber scanning apparatuses 10E to 10H are similar to the optical fiber scanning apparatus 10D, components having the same functions are denoted by the same reference numerals and signs and explanation of the components is omitted.

The optical fiber scanning apparatuses 10E to 10H have the effects of the optical fiber scanning apparatus 10D and further have characteristic effects.

### Modification 1 of the Second Embodiment

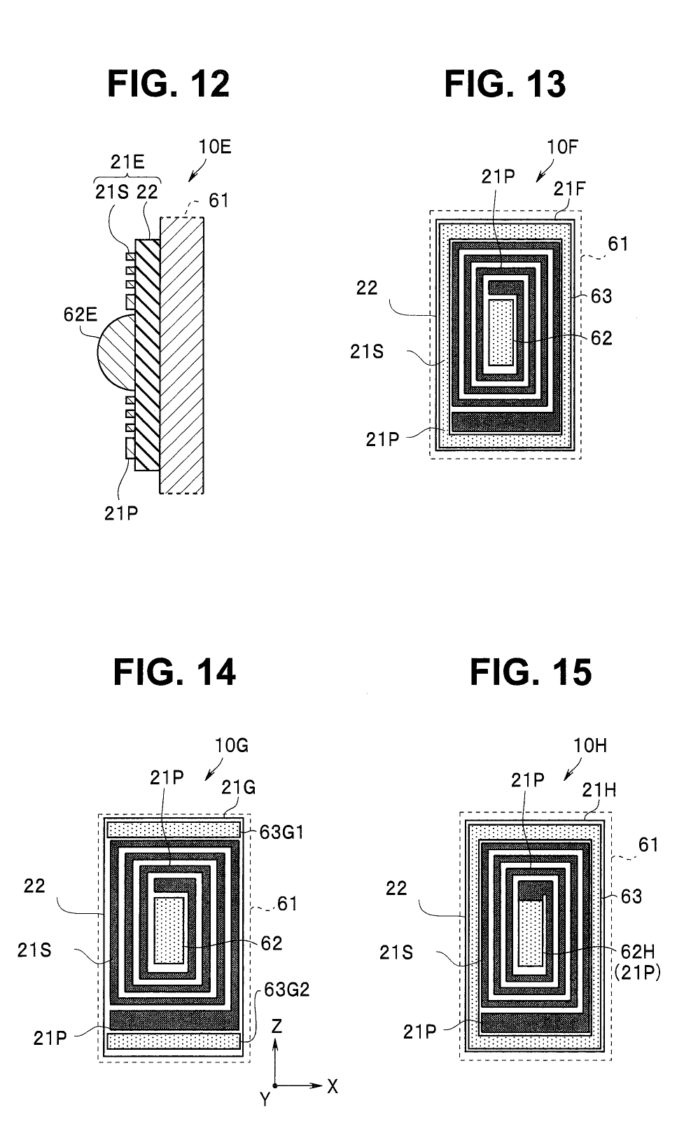

As shown in FIG. 12, in the optical fiber scanning apparatus 10E in the modification 1, a core 62E has a convex shape toward the center axis O. A distal end of the core 62E has a hemispherical shape. In other words, a sectional area of the core 62E decreases further away from a coil chip 21E (the outer yoke 61D).

In a rectangular parallelepiped core, since the magnetic field M concentrates on corner portions (edge portions), it is likely that a magnetic body is partially in a saturated state and the magnetic field cannot be inducted. On the other hand, in the optical fiber scanning apparatus 10E including the core 62E that has the convex shape and the distal end of which has the hemispherical shape, since the core 62E is less easily locally saturated, it is possible to perform efficient scan irradiation.

### Modification 2 of the Second Embodiment

As shown in FIG. 13, the optical fiber scanning apparatus 10F in the modification 2, an inner magnetic-field inducing section further includes, in addition to the core 62, a frame-like inner yoke 63 annularly disposed around the plane coil 21S of a coil chip 21F.

In the optical fiber scanning apparatus 10F, since a magnetic field is induced by the inner yoke 63, the magnetic field M generated by the plane coil 21S less easily leaks to the outside of the magnetic-field generating unit 21U. It is possible to concentrate the magnetic field M on the vicinity of the permanent magnet 31.

### Modification 3 of the Second Embodiment

As shown in FIG. 14, the optical fiber scanning apparatus 10G in the modification 3 includes two inner yokes 63G1 and 63G2 disposed along two sides orthogonal to the center axis O of the housing 11 in a periphery of the plane coil 21S rectangular in plan view of a coil chip 21G.

As shown in FIG. 3, since the coil chip 21 is disposed in the narrow hollow section 11H on the inside of the housing 11, a space for disposing an inner yoke extended in a direction parallel to the center axis O of the housing 11 (a Z-axis direction) sometimes cannot be secured in the coil chip 21.

However, the coil chip 21G of the optical fiber scanning apparatus 10G includes two inner yokes 63G1 and 63G2 extended in a direction orthogonal to the center axis O (the X-axis direction) in which a space for disposing the inner yokes 63G1 and 63G2 are easily secured.

In the optical fiber scanning apparatus 10G, as in the optical fiber scanning apparatus 10F, the magnetic field M generated by the plane coil 21S less easily leaks to the outside of the magnetic-field generating unit 21U. It is possible to concentrate the magnetic field M on the vicinity of the permanent magnet 31.

### Modification 4 of the Second Embodiment

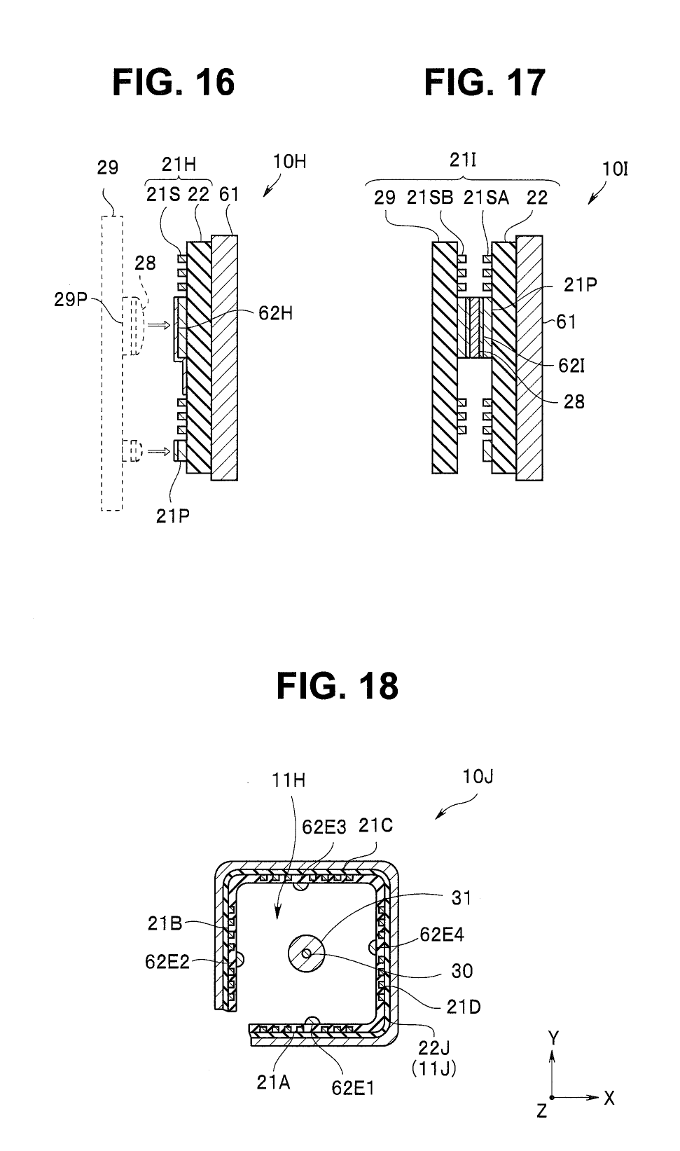

As shown in FIG. 15 and FIG. 16, in the optical fiber scanning apparatus 10H in the modification 4, a core 62H disposed in the center portion of the plane coil 21S of a coil chip 21H is disposed on the bonding pad 21P of the plane coil 21S. That is, the core 62H is made of a conductive soft magnetic material such as nickel or a permalloy. A driving current flows to the core 62H.

As shown in FIG. 16, the bonding pad 21P is bonded to the electrode 29P of the flexible wiring board 29 by solder 28. For example, the core 62H having a film thickness of 3 μm made of a permalloy plated film has a function of a solder barrier layer of the electrode 29P made of copper, gold, or the like. In order to have the function of the solder barrier layer, the core 62H desirably includes at least 50 wt % or more of nickel.

As in the coil chip 21G, it is also possible to form an inner yoke on the flexible wiring board 29 from a soft magnetic foil or the like.

In the optical fiber scanning apparatus 10H, since the core 6214 has the function of the solder barrier layer, it is unnecessary to dispose the solder barrier layer. Therefore, it is easy to manufacture the optical fiber scanning apparatus 10H.

### Third Embodiment

An optical fiber scanning apparatus 10I in a third embodiment is explained. Since the optical fiber scanning apparatus 10I is similar to the optical fiber scanning apparatus 10 and the like, components having the same functions are denoted by the same reference numerals and signs and explanation of the components is omitted.

As shown in FIG. 17, a coil 21SI of a coil chip 21I of the optical fiber scanning apparatus 10I is a multilayer coil in which a first plane spiral coil 21SA and a second plane spiral coil 21SB are superimposed. At least a part of a connection body disposed in a center portion that electrically connects the first plane spiral coil 21SA and the second plane spiral coil 21SB is a core 621 made of a conductive soft magnetic material.

The optical fiber scanning apparatus 10I has the functions of the optical fiber scanning apparatus 10H and the like. Further, the coil 21SI is the multilayer coil. Therefore, the optical fiber scanning apparatus 10I can be driven by a smaller electric current. Note that the optical fiber scanning apparatus in this modification may include a multilayer coil in which three or more layers of coils are stacked.

### Fourth Embodiment

An optical fiber scanning apparatus 10J in a fourth embodiment is explained. Since the optical fiber scanning apparatus 10J is similar to the optical fiber scanning apparatus 10 and the like, components having the same functions are denoted by the same reference numerals and signs and explanation of the components is omitted.

As shown in FIG. 18, in the optical fiber scanning apparatus 10J, a housing is configured by a common substrate 22J on which the coil chips 21A to 21D are disposed. The substrate 22J is made of, for example, polyimide having flexibility. The substrate 22J is bent to configure a housing 11J including the hollow section 11H.

Cores 62E (62E1 to 62E4) having a convex shape are disposed in center portions of the respective coil chips 21A to 21D.

The optical fiber scanning apparatus 10J has the effects of the optical fiber scanning apparatus 10H and the like. Further, since the substrate 22J configures the housing, the optical fiber scanning apparatus 10J is small in diameter.

Note that, if the coil chips 21A to 21D including a nonflexible substrate 22 made of silicon are consecutively connected by the insulating layer 23, it is possible to configure the housing by bending the coil chips 21A to 21D via the insulating layer 23.

### Fifth Embodiment

Optical scanning endoscopes (endoscopes) 2 and 2A to 2J in a fifth embodiment are explained.

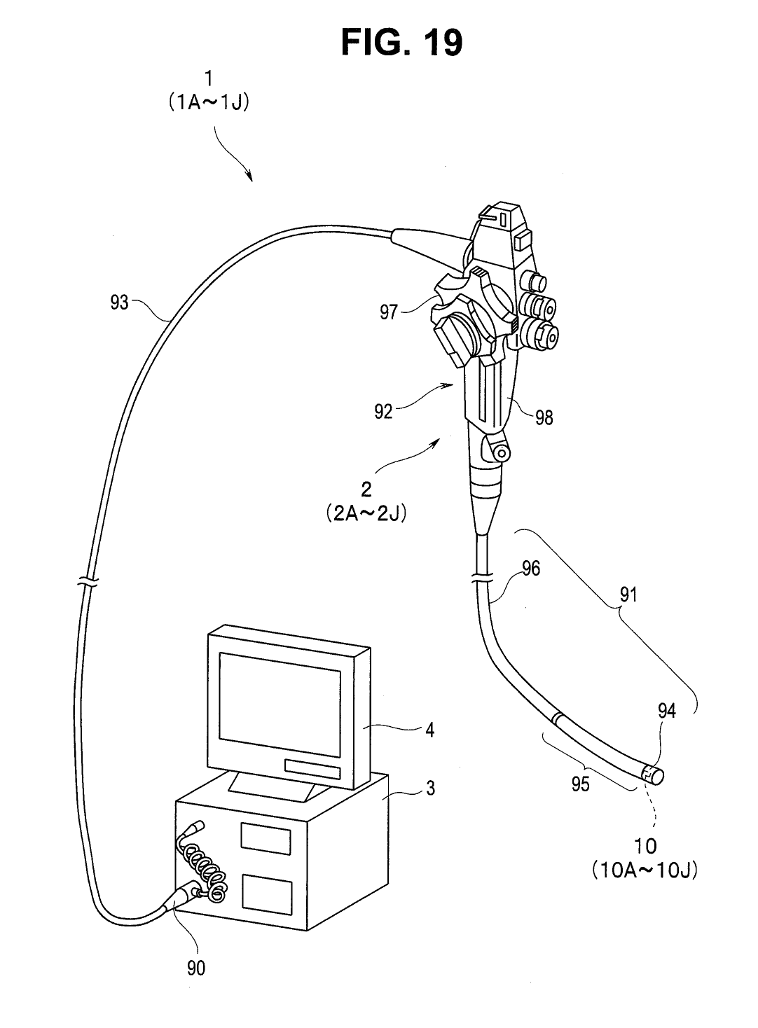

As shown in FIG. 19, the optical scanning endoscopes 2 and 2A to 2J include, at a distal end portion 94 of an insertion section 91, the optical fiber scanning apparatuses 10 and 10A to 10J explained above. The endoscope 2 including the optical fiber scanning apparatus 10 is explained as an example below.

An optical scanning endoscope system (hereinafter referred to as “endoscope system”) 1 including the endoscope 2 includes the endoscope 2, a main body apparatus 3 including functions of a light sourced apparatus and a video processor, and a monitor 4. The endoscope 2 irradiates illumination light on a subject while two-dimensionally scanning the illumination light with the optical fiber scanning apparatus 10, detects reflected light (return light) from the subject, performs data processing in the main body apparatus 3, and displays a generated subject image on the monitor 4.

The endoscope 2 includes an elongated insertion section 91 inserted through a living organism, an operation section 92, and a universal cable 93 through which an electric cable and the like are inserted. The insertion section 91 of the endoscope 2 includes a distal end portion 94, a bending section 95, and a flexible tube section 96. Note that the endoscope 2 in the embodiment is a so-called flexible endoscope. However, a so-called rigid endoscope, the insertion section 91 of which is rigid, also has effects explained below.

In the operation section 92, a bending operation knob 97 for bending the bending section 95 is turnably disposed. A coupling section of the insertion section 91 and the operation section 92 is a grasping section 98 grasped by a user.

The universal cable 93 extended from the operation section 92 is connected to the main body apparatus 3 via a connector 90. The main body apparatus 3 is connected to the monitor 4 that displays an endoscopic image.

The endoscope 2 includes, at a distal end portion of an insertion section, an optical fiber scanning apparatus including a tubular housing, an optical fiber disposed along a center axis of the housing, provided with a permanent magnet, and configured to emit light from a free end, and a coil disposed in the housing and configured to apply a magnetic field to the permanent magnet and drive the free end of the optical fiber. The optical fiber scanning apparatus includes, on an inside, which is the center axis side of the housing, an outside opposite to the inside, or the inside and the outside, a magnetic-field inducing section made of a soft magnetic material configured to induce the magnetic field generated by the coil.

The endoscopes 2 and 2A to 2J include the optical fiber scanning apparatuses 10 and 10A to 10J that perform efficient scan irradiation in which a magnetic field less easily leaks.

The present invention is not limited to the respective embodiments explained above. It goes without saying that various changes, combinations, and applications are possible in a range not departing from the spirit of the invention.

What is claimed is:

1. An optical fiber scanning apparatus comprising:
a tubular housing;
an optical fiber disposed along a Z axis, which is a center axis of the housing, provided with a permanent magnet, and configured to emit light from a free end thereof;
an X-axis coil including at least one spiral coil configured to apply a magnetic field in an X-axis direction orthogonal to the Z axis to the permanent magnet and scan the free end in the X-axis direction;
a Y-axis coil including at least one spiral coil configured to apply a magnetic field in a Y-axis direction orthogonal to the Z axis and the X axis to the permanent magnet and scan the free end in the Y-axis direction; and
a magnetic-field inducing section including an inner magnetic-field inducing section including a core disposed in a center portion on an inside, which is the center axis side of each of the spiral coils included in the x-axis coil and the y-axis coil, and configured to induce the magnetic field in a direction perpendicular to the center axis of the housing and an outer magnetic-field inducing section disposed on an outside opposite to the inside of each of the spiral coils.

2. The optical fiber scanning apparatus according to claim 1, wherein the outer magnetic-field inducing section covers one surface of each of the spiral coils.

3. The optical fiber scanning apparatus according to claim 2, wherein at least a part of the housing is made of a soft magnetic material and configures the outer magnetic-field inducing section.

4. The optical fiber scanning apparatus according to claim 1, wherein each of the X-axis coil and the Y-axis coil includes two spiral coils disposed to be opposed to each other across the center axis.

5. The optical fiber scanning apparatus according to claim 1, wherein the core has a convex shape toward the center axis, a distal end of the core having a hemispherical shape.

6. The optical fiber scanning apparatus according to claim 1, wherein the inner magnetic-field inducing section further includes an inner yoke annularly disposed around the spiral coil.

7. The optical fiber scanning apparatus according to claim 1, wherein the inner magnetic-field inducing section includes two inner yokes disposed along two sides orthogonal to the center axis of the housing in a periphery of the spiral coil rectangular in plan view.

8. The optical fiber scanning apparatus according to claim 1, wherein the spiral coil is a multilayer coil in which a first plane spiral coil and a second plane spiral coil are superimposed, and at least a part of a connection body disposed in the center portion that electrically connects the first plane spiral coil and the second plane spiral coil is the core.

9. The optical fiber scanning apparatus according to claim 1, wherein the housing is configured by a substrate on which the spiral coil is disposed.

10. An optical scanning endoscope including the optical fiber scanning apparatus according to claim 1 at a distal end portion of an insertion section thereof.
